(19) United States
(12) Patent Application Publication
(10) Pub. No.: US 2013/0053878 A1
(43) Pub. Date: Feb. 28, 2013

(54) CAP ASSEMBLY FOR ALTERNATE SITE TESTING AND/OR UTILIZING A LOCKING SYSTEM AND/OR LOCKED DEPTH OF PENETRATION POSITIONS AND/OR LANCET DEVICE USING SUCH FEATURES

(71) Applicant: Steven SCHRAGA, Surfside, FL (US)

(72) Inventor: Steven SCHRAGA, Surfside, FL (US)

(73) Assignee: STAT MEDICAL DEVICES, INC., N. Miami Beach, FL (US)

(21) Appl. No.: 13/594,275

(22) Filed: Aug. 24, 2012

Related U.S. Application Data
(60) Provisional application No. 61/527,909, filed on Aug. 26, 2011.

(51) Int. Cl. A61B 17/34

(52) U.S. Cl. 606/181

(57) ABSTRACT

The invention provides for a lancet device and/or a cap assembly for a lancet device which includes a member capable of being removably mounted to a body of the lancet device. An alternate site testing (AST) cap can be mounted to the member. A method of using the lancet device includes mounting the member to a body of the lancet device, placing the alternate site testing (AST) cap against a skin surface, and forming a puncture in the skin surface with the needle. A method the lancet device includes mounting the member to a body of the lancet device and mounting the alternate site testing (AST) cap onto the member.

### CROSS-REFERENCE TO RELATED APPLICATIONS

The instant application is a U.S. non-provisional application that is based on and claims the benefit of U.S. provisional application No. 61/527,909, filed Aug. 26, 2011, the disclosure of which is hereby expressly incorporated by reference thereto in its entirety.

### BACKGROUND OF THE INVENTION

1. Field of the Invention

The invention relates to a cap assembly which utilizes one or more features shown in the drawings and/or recited in the claims. A method of making and using the cap assembly and/or lancet device is also disclosed.

2. Discussion of Background Information

Lancet devices are commonly used to prick the skin of the user so that one or more drops of blood may be extracted for testing. Some users, such as diabetics, for example, may have to test their blood sugar levels several times a day. This may be accomplished by the user using a simple needle. However, this procedure is often problematic for the user since the needle may be difficult to handle. Additionally, many users simply cannot perform the procedure owing to either a fear of needles or because they lack a steady hand. As a result, lancet devices have been developed which allow the user to more easily and reliably perform this procedure.

Most lancet devices lack convenient and flexible adjustability. Such devices are typically made adjustable by switching their tips. U.S. Pat. No. Re. 32,922 to LEVIN et al. is one such device. That is, the user must remove one tip having a set depth and replace it with another having a different set depth. This, of course, creates the problem of storing the replaceable tips, which if not properly done, may result in their misplacement, damage, contamination, or the like.

An improved device would allow the user to more easily adjust the depth of penetration and would overcome some of the disadvantages described above. Moreover, since the skin thickness can vary slightly from user to user and finger to finger, a need exists for efficiently adapting the depth of penetration. For example, an index finger may be more calloused than a middle finger, and the more calloused finger will typically have thicker skin. By adjusting the depth of puncture so that the depth is no greater than necessary for extracting a required amount of blood, any pain experienced by the user may be minimized.

Lancets having an adjustable tip are known per se. For example, U.S. Pat. No. 4,469,110 to SLAMA discloses a mechanism which adjusts the penetration depth by rotating a threaded sleeve relative to a body. The SLAMA device is characterized as a “single bottom” device which employs a threaded design which can be expensive to manufacture. Further, such a threaded resign is prone to inadvertent setting changes since there is nothing but frictional engagement between the mating threads to maintain the adjustment setting.

U.S. Pat. No. 4,895,147 to BODICKY et al. functions in a similar manner to the device in SLAMA and therefore suffers from similar disadvantages.

U.S. Pat. Nos. 5,464,418, 5,797,942, 5,908,434, 6,156,051 and 6,530,937 to SCHRAGA also disclose similar lancet devices and are hereby incorporated herein by reference as though set forth in full herein.

As disclosed in U.S. Pat. No. 5,908,434, the lancet device has a body portion which encloses a lancet and a lancet firing mechanism. The lancet typically has a needle extending therefrom and is caused to move towards the tip of the device by a trigger or firing mechanism. The lancet device forces the needle, by virtue of the needle being fixed thereto, out of the device by some distance or depth so that the needle can penetrate the skin of the user. The function of this firing mechanism and the lancet body design is disclosed in each of U.S. Pat. No. 5,797,942 and U.S. Pat. No. 5,908,434. These patents are incorporated by reference herein in their entirety and are therefore only briefly discussed herein. Similarly, U.S. Pat. No. 6,156,051 discloses a lancet device which utilizes a lancet firing mechanism, a depth adjustment mechanism, and a trigger setting mechanism. This patent is incorporated by reference herein in its entirety.

Devices for Alternate Site Testing (AST) also known in the art. For example, U.S. Pat. No. 6,491,709 to SHARMA et al., US 2004/0024553 to MONFRE et al., and US 2004/0236251 to ROE et al. teach AST lancet devices or methods and are each hereby entirely incorporated herein by reference as though set forth in full herein. Such devices, however, lack the combination of features of the invention. A lancet device used for alternate site testing typically forms a skin puncture on parts of the body other than a finger tip. The areas most frequently used as alternative sites for blood samples include the palm, forearm and thigh.

Current lancets utilize a needle length of about 3.3 mm plus or minus some tolerance.

Thus, while advances have been made, there is a continuing need for a lancet device which provides for convenient, reliable and easy adjustment of penetration depth and especially in the context of AST.

### SUMMARY OF THE INVENTION

According to non-limiting embodiments of the invention, there is provided a lancet device comprising a cap assembly comprising a member capable of being removably mounted to a body of the lancet device and an AST cap mounted to the member. A lancet is utilized having a needle that is greater than 3.3 mm long can be utilized, and preferably the length is at least about 3.4 mm long, more preferably it is about 3.5 mm or greater, and most preferably it is greater than 3.5 mm.

In embodiments, the member comprises a stop surface adapted to be contacted by a front end of the lancet.

In embodiments, the AST cap is movable relative to the member between at least two axial positions. In embodiments, the at least two axial positions define different depth of penetrations of the needle.

In embodiments, the member comprises a stop surface adapted to be contacted by a front end of the lancet.

In embodiments, the AST cap is movable relative to the member between plural axial positions.

In embodiments, the AST cap is rotatable relative to the member between plural positions.

In embodiments, the member comprises a stop surface adapted to be contacted by a front end of the lancet.

In embodiments, the lancet is removably mountable to a holding member of the lancet device.

In embodiments, the cap assembly utilizes depth of penetration adjustment.

In embodiments, the device further comprises a second cap assembly adapted to be removably mounted to the body of the lancet device.

In embodiments, the second cap assembly utilizes depth of penetration adjustment.

In embodiments, the second cap assembly is a non-AST cap assembly.

In embodiments, the body of the lancet device utilizes depth of penetration adjustment.

In embodiments, the body comprises a stop surface for limiting forward travel of a lancet holding member movably arranged within the body.

In embodiments, the body includes therein a stop surface for limiting forward travel of a lancet holding member movably arranged within the body.

In embodiments, the lancet device utilizes depth of penetration adjustment.

In embodiments, the depth of penetration adjustment is determined by a stop surface for limiting forward travel of a lancet holding member and a skin engaging surface of the AST cap.

In embodiments, the AST cap is adjustably movable between at least two depth of penetration positions, a first of the at least two depth of penetration positions being spaced from the stop surface by a lesser amount than a second of the at least two depth of penetration positions.

According to non-limiting embodiments of the invention, there is provided a lancet device comprising a cap assembly one of comprising an alternate site testing (AST) cap and a non-AST cap each mountable to a member and two separate caps each being installable on the lancet device wherein one of the caps is an AST cap mounted to a member and another of the caps is a non-AST cap mounted to a member. The member is capable of being removably mounted to a body of the lancet device.

In embodiments, the device further comprises a lancet having a needle that is at least 3.5 mm long.

In embodiments, the member comprises a stop surface adapted to be contacted by a front end of the lancet.

In embodiments, the AST cap is movable relative to the member between at least two axial positions.

In embodiments, the at least two axial positions define different depth of penetrations of the needle.

In embodiments, the member comprises a stop surface adapted to be contacted by a front end of the lancet.

In embodiments, the AST cap is movable relative to the member between plural axial positions.

In embodiments, the AST cap is rotatable relative to the member between plural positions.

In embodiments, the member comprises a stop surface adapted to be contacted by a front end of the lancet.

In embodiments, the lancet is removably mountable to a holding member of the lancet device.

In embodiments, the cap assembly utilizes depth of penetration adjustment.

In embodiments, the body of the lancet device utilizes depth of penetration adjustment.

In embodiments, the body comprises a stop surface for limiting forward travel of a lancet holding member movably arranged within the body.

In embodiments, the body includes therein a stop surface for limiting forward travel of a lancet holding member movably arranged within the body.

In embodiments, the lancet device utilizes depth of penetration adjustment.

In embodiments, the depth of penetration adjustment is determined by a stop surface for limiting forward travel of a lancet holding member and a skin engaging surface of the AST cap.

In embodiments, the AST cap is adjustably movable between at least two depth of penetration positions, a first of the at least two depth of penetration positions being spaced from the stop surface by a lesser amount than a second of the at least two depth of penetration positions.

According to non-limiting embodiments of the invention, there is provided a lancet device comprising a cap assembly comprising a member capable of being removably mounted to a body of the lancet device and an alternate site testing (AST) cap capable of being mounted to the member. A non-AST cap is utilized that is capable of being mounted to the member.

In embodiments, the device further comprises a lancet having a needle that is at least 3.5 mm long.

In embodiments, the invention provides for a cap assembly for a lancet device, wherein the cap assembly comprises a member capable of being removably mounted to a body of the lancet device, an alternate site testing (AST) cap capable of being mounted to the member, and a non-AST cap capable of being mounted to the member.

In embodiments, the cap assembly further comprises a lancet having a needle that is at least 3.5 mm long.

In embodiments, the invention provides for a method of using any of the lancet devices described above, wherein the method comprises mounting the member to a body of the lancet device, placing the alternate site testing (AST) cap against a skin surface, and forming a puncture in the skin surface with the needle.

In embodiments, the invention provides for a method of using any of the lancet devices described above, wherein the method comprises mounting the member to a body of the lancet device, placing the alternate site testing (AST) cap against a skin surface, and forming a puncture in the skin surface.

In embodiments, the invention provides for a method of making any of the lancet devices described above, wherein the method comprises mounting the member to a body of the lancet device and mounting the alternate site testing (AST) cap onto the member.

In embodiments, the invention provides for a method of making any of the lancet devices described above, wherein the method comprises mounting the member to a body of the lancet device and mounting the alternate site testing (AST) cap onto the member.

In embodiments, each lancet device embodiment disclosed in U.S. Pat. No. 5,464,418 to SCHRAGA, the entire disclosure of which is herein expressly incorporated by reference, is modified to utilize one or more of the cap assembly embodiments disclosed herein.

In embodiments, each lancet device embodiment disclosed in U.S. Pat. No. 5,908,434 to SCHRAGA, the entire disclosure of which is herein expressly incorporated by reference, is modified to utilize one or more of the cap assembly embodiments disclosed herein.

In embodiments, each lancet device embodiment disclosed in U.S. Pat. No. 6,022,366 to SCHRAGA, the entire disclosure of which is herein expressly incorporated by reference, is modified to utilize one or more of the cap assembly embodiments disclosed herein.

In embodiments, each lancet device embodiment disclosed in U.S. Pat. No. 7,905,898 to SCHRAGA, the entire disclosure of which is herein expressly incorporated by reference, is modified to utilize one or more of the cap assembly embodiments disclosed herein.

In embodiments, each lancet device embodiment disclosed in U.S. Pat. No. 7,621,931 to SCHRAGA, the entire disclosure of which is herein expressly incorporated by reference, is modified to utilize one or more of the cap assembly embodiments disclosed herein.

In embodiments, each lancet device embodiment disclosed in US 2010/0274273 to SCHRAGA, the entire disclosure of which is herein expressly incorporated by reference, is modified to utilize one or more of the cap assembly embodiments disclosed herein.

In embodiments, each lancet device embodiment disclosed in US 2008/0195132 to SCHRAGA, the entire disclosure of which is herein expressly incorporated by reference, is modified to utilize one or more of the cap assembly embodiments disclosed herein.

In embodiments, each lancet device embodiment disclosed in US 2007/0083222 to SCHRAGA, the entire disclosure of which is herein expressly incorporated by reference, is modified to utilize one or more of the cap assembly embodiments disclosed herein.

In embodiments, each lancet device embodiment disclosed in US 2006/0241668 to SCHRAGA, the entire disclosure of which is herein expressly incorporated by reference, is modified to utilize one or more of the cap assembly embodiments disclosed herein.

In embodiments, each lancet device embodiment disclosed in US 2006/0173478 to SCHRAGA, the entire disclosure of which is herein expressly incorporated by reference, is modified to utilize one or more of the cap assembly embodiments disclosed herein.

In embodiments, there is provided a cap assembly for a lancet device comprising at least one of a member capable of being removably mounted to a body of the lancet device and a cap mounted to the member and moveble between at least two locked positions and at least one cap having at least two lockable depth of penetration adjustment positions.

In embodiments, there is provided a lancet device comprising at least one of a member capable of being removably mounted to the lancet device and a cap mounted to the member and movable between at least two locked positions and a mechanism for locking a cap in at least two depth of penetration adjustment positions.

In embodiments, there is provided a cap assembly for a lancet device comprising at least one of a member capable of being removably mounted to a body of the lancet device and a cap mounted to the member and having at least the following modes of operation, an adjusting mode allowing a user to at least one of adjust a depth of penetration position of a skin engaging surface of the cap, change an axial distance between a skin engaging surface of the cap, and move the cap between at least two depth of penetration positions, and a locking mode wherein the user is prevented from adjusting the depth of penetration until the user places the cap in the unlocked position.

In embodiments, there is provided a lancet device comprising a member capable of being mounted to the lancet device and a cap mounted to the member and the cap being moveable between at least two depth of penetration positions when in an unlocked position and being prevented from moving between the at least two depth of penetration positions when in the locked position.

In embodiments, there is provided a lancet device comprising at least two depth of penetration positions and being lockable in at least one of the at least two depth of penetration positions via a locking device.

In embodiments, there is provided a lancet device with depth of penetration adjustment and being lockable in at least one depth of penetration position via a locking device.

In embodiments, there is provided a lancet device with depth of penetration adjustment having at least one puncturing position and at least one non-puncturing position.

In embodiments, there is provided a lancet device comprising at least the following modes of operation: a mode wherein a skin engaging surface is automatically disposed in a position that prevents puncturing of a user's skin when the lancet device is triggered; and a mode wherein the skin engaging surface is disposed in a position that allows puncturing of a user's skin when the lancet device is triggered.

In embodiments, there is provided a lancet device with depth of penetration adjustment comprising at least the following modes of operation: a mode wherein a skin engaging surface is automatically disposed in a position that prevents puncturing of a user's skin when the lancet device is triggered; and a mode wherein the skin engaging surface is disposed in a position that allows puncturing of a user's skin when the lancet device is triggered.

In embodiments, there is provided a lancet device comprising at least the following modes of operation: a mode wherein the skin engaging surface is biased towards a position that prevents puncturing of a user's skin when the lancet device is triggered; and a mode wherein the skin engaging surface is movable to a position that allows puncturing of a user's skin when the lancet device is triggered.

In embodiments, there is provided a lancet device comprising at least the following modes of operation: a default mode wherein the skin engaging surface is in a position that prevents puncturing of a user's skin when the lancet device is triggered; and a puncturing mode wherein the skin engaging surface is selectively movable to a position that allows puncturing of a user's skin when the lancet device is triggered.

In embodiments, there is provided a cap assembly for a lancet device comprising at least the following modes of operation: a default mode wherein the skin engaging surface is in a position that prevents puncturing of a user's skin when the lancet device is triggered; and a puncturing mode wherein the skin engaging surface is selectively movable to a position that allows puncturing of a user's skin when the lancet device is triggered.

In embodiments, the devices of anyone of the above-noted devices further comprise at least one of an alternate site testing (AST) cap, a non-AST cap, a depth of penetration adjustment system, and a locking system for locking the cap assembly in one of said modes of operation.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side cross-section view of a cap assembly in accordance with the invention. The cap is shown in an intermediate axial and rotational depth of penetration position;

FIG. 2 shows the cap assembly of FIG. 1 with the cap being shown in an minimum depth of penetration position;

FIG. 3 shows the cap assembly of FIG. 1 with the cap being shown in a maximum depth of penetration position;

FIG. 4 shows the cap assembly of FIG. 1 installed on a body or housing of a lancet device;

FIG. 5 shows the device of FIG. 4 in a puncturing configuration;

FIG. 6 shows the cap assembly removed from the lancet device of FIG. 4;

FIG. 7 shows the lancet device of FIG. 4 with the cap assembly removed;

FIG. 8 shows a lancet used in the lancet device of FIG. 4;

FIGS. 9 and 10 show side cross-section views of another embodiment of a cap assembly in accordance with the invention. In this embodiment, the lancet device can utilize one of the two cap assemblies shown in FIGS. 9 and 10. The cap assembly of FIG. 9 is used on the lancet device when the user wants to puncture skin using a non-AST cap and the cap assembly of FIG. 10 is used on the lancet device when the user wants to puncture skin using an AST cap. Both cap assemblies utilize depth of penetration adjustment;

FIG. 11 shows a side cross-section view of an embodiment of a cap assembly arranged on a lancet device in accordance with the invention. In this embodiment, the lancet device utilizes a stop arranged in the body and depth of penetration adjustment on the cap assembly;

FIGS. 12 and 13 show side cross-section views of another embodiment of a cap assembly in accordance with the invention. In this embodiment, the cap assembly can utilize one of the two caps shown in FIGS. 12 and 13. The cap shown in FIG. 12 is placed on the cap assembly member when the user wants to puncture skin using a non-AST cap and the cap shown in FIG. 10 is used on the same cap assembly member when the user wants to puncture skin using an AST cap. Both cap assemblies utilize a depth of penetration adjustment;

FIG. 14 shows the cap assembly member arranged on the lancet device as shown in FIGS. 12 and 13 and without either the non-AST cap or the AST cap installed thereon;

FIG. 15 shows the AST cap usable on the cap assembly member shown in FIG. 14;

FIG. 16 shows the non-AST cap usable on the cap assembly member shown in FIG. 14;

FIGS. 17 and 18 show different configurations of lancets which can be utilized with the invention and shows how the length of the lancet needle is measured;

FIGS. 19-22 show various views of another embodiment of a cap assembly in accordance with the invention. In this embodiment, the cap assembly provides for a cap which can be locked in different depth of penetration positions. In FIG. 19, the cap is shown locked in one of the positions whereas FIG. 20 shows the cap is an unlocked position. Only in the unlocked position of FIG. 20 can the user rotate the cap relative to the member. FIG. 21 shows a portion of the member having locking recesses. FIG. 22 shows the cap used in this embodiment with an optional gripping surface;

FIGS. 23 and 24 show various views of another embodiment of a cap assembly in accordance with the invention. In this embodiment, the cap assembly is similar to the previous embodiment except that it utilizes an AST cap instead of a non-AST cap;

FIGS. 25 and 26 show side cross-section views of another embodiment of a cap assembly in accordance with the invention. In this embodiment, the cap assembly utilizes a locking system to either allow depth of penetration adjustment or prevent it. In FIG. 25, the locking system is not engaged or is in the unlocked position and allows a user to adjust depth of penetration. In FIG. 26, the locking system is engaged or is in the locked or locking position and prevents a user from adjusting depth of penetration;

FIGS. 27 and 28 shows cross-section and bottom view of the locking member used on the embodiment of FIGS. 25 and 26;

FIG. 29 shows a top view of the cap used in the embodiment of FIGS. 25 and 26;

FIG. 30 shows a cross-section view of another embodiment of the invention;

FIG. 31 shows a cross-section view of another embodiment of the invention with the cap being disposed in a default safety mode;

FIG. 32 shows a cross-section view of the embodiment of FIG. 31 with the cap being disposed in a skin puncturing mode;

FIG. 33 shows a cross-section view of a non-AST cap used in the embodiment of FIG. 31 and shows how the spring is arranged therein;

FIG. 34 shows a cross-section view of a non-AST cap used in the embodiment of FIG. 31 and shows the spring before being installed therein; and

FIG. 35 shows a cross-section view of an AST cap that can be used in the embodiment of FIG. 31 and shows the spring before being installed therein.

### DETAILED DESCRIPTION OF THE INVENTION

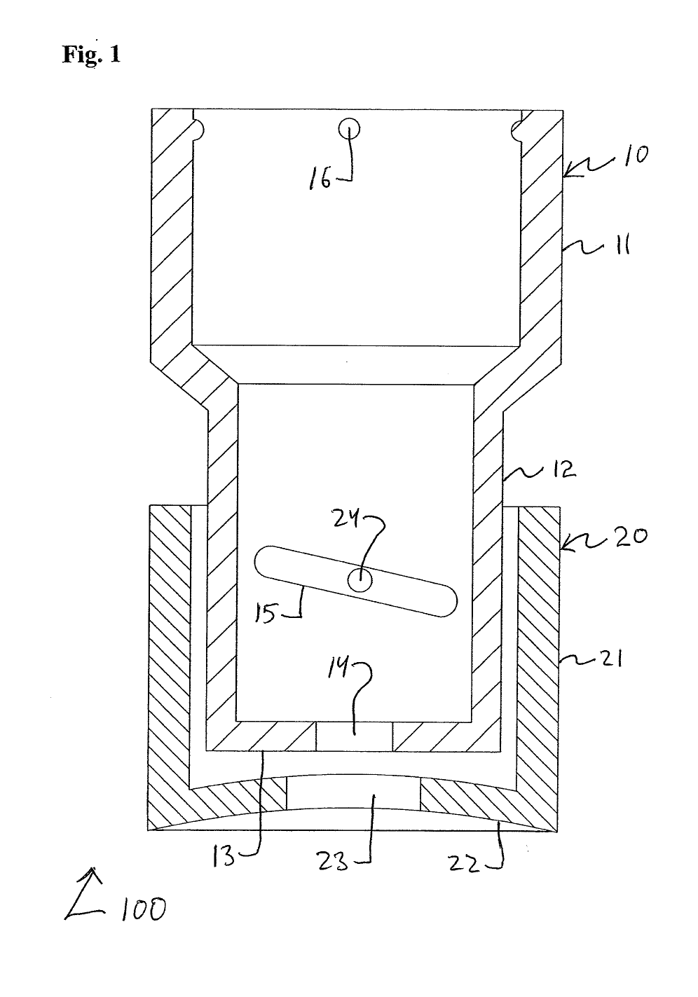

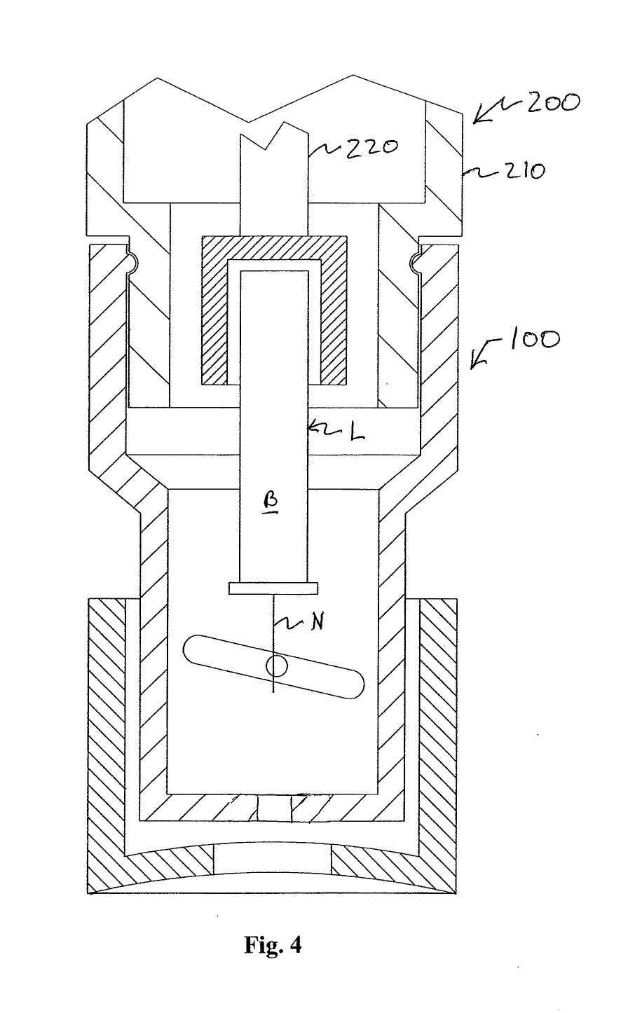

FIGS. 1-3 show a first non-limiting embodiment of the invention. In this first embodiment, a cap assembly 100 includes a member 10 capable of being removably mounted to a body of the lancet device as well as an alternate site testing (AST) cap 20 mounted to the member 10. As will be described later on with reference to FIGS. 4-8, the cap assembly 100 can be utilized with a lancet device 200 having a lancet L whose needle N is at least 3.5 mm long.

Again referring to FIGS. 1-3, it can be seen that the member 10 has a distal or rear body portion 11 and a proximal portion 12. The rear body portion 11 is configured to be mounted or removably mounted to a lancet device. One or more projections 16 can be utilized to ensure that the portion 11 is releasably retained on a body of the lancet device. The front body portion 12 is configured so that the cap 20 can be mounted or rotatably mounted thereto. In order to provide depth adjustment, a projection 24 of the cap 20 projects radially inwardly from the sidewall 21 and is sized and configured to extend into and slidably engage with an angled or helically oriented slot 15 arranged on or in the front portion 12. End wall 13 of the member 10 includes a lancet opening 14 which is sized and configured to allow the needle of the lancet to pass therethrough, but not the body of the lancet. Skin contacting wall 22 of the member 20 includes a lancet opening 23 which is sized and configured to allow the needle of the lancet to pass therethrough and puncture skin.

In embodiments, each of the member 10 and the cap 20 is a one-piece or integrally formed synthetic resin member made of a material typically utilized in caps for lancet devices. In embodiments, walls 11, 12 and 21 are generally cylindrical while wall 13 is generally planar and circular. In embodiments, wall 22 is generally inwardly curved and circular.

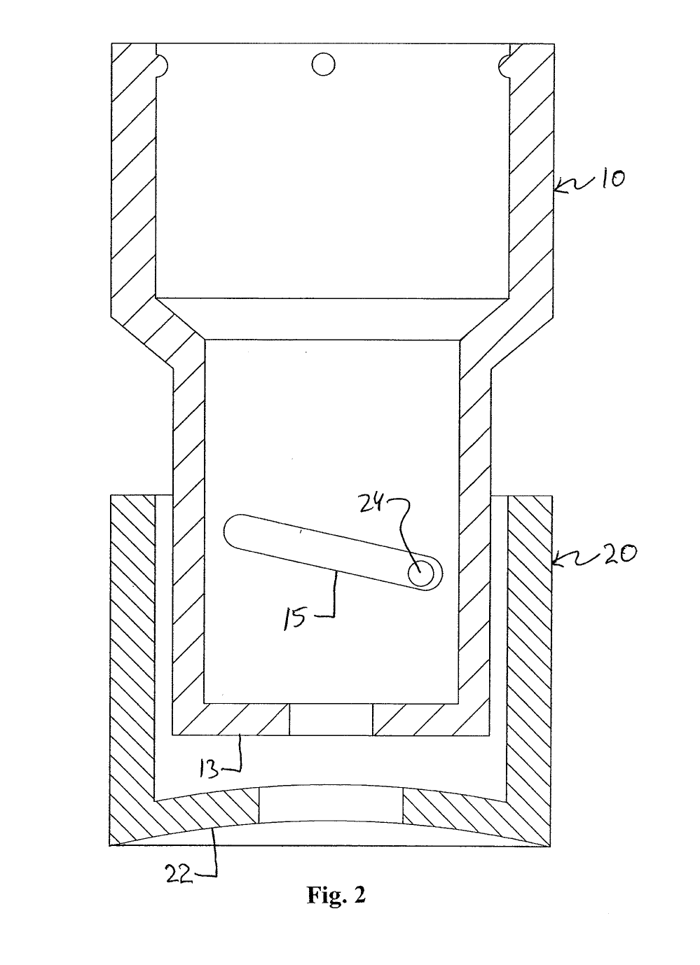

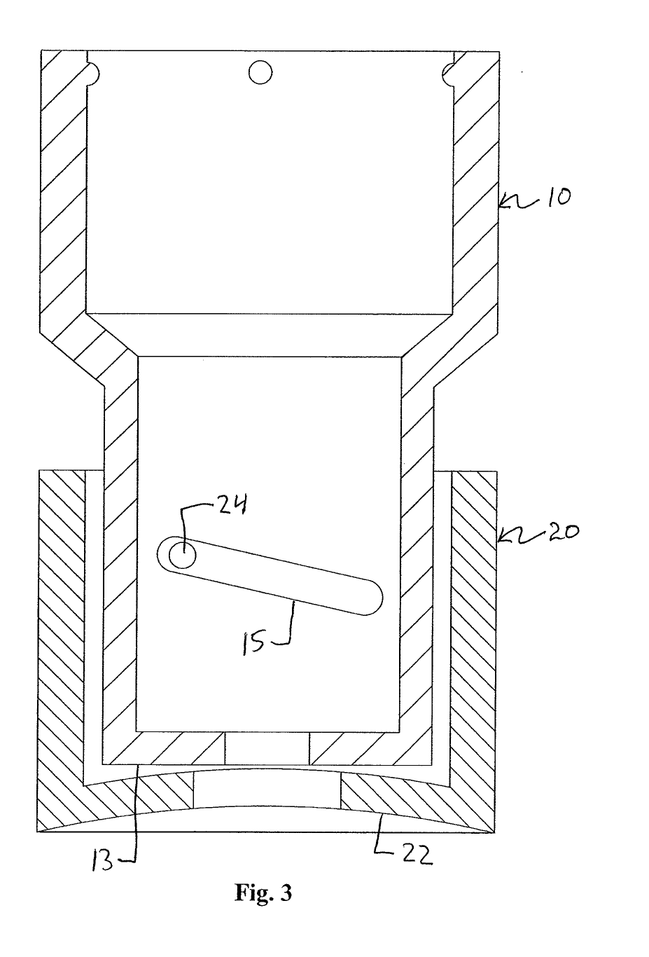

FIG. 1 shows the cap 20 in an intermediate axial and rotational depth of penetration position with the projection 24 generally arranged in a middle portion of the slot 15. FIG. 2 shows the cap 20 in a minimum depth of penetration position with the projection 24 generally arranged in one of the two end portions of the slot 15. FIG. 3 shows the cap 20 in a maximum depth of penetration position with the projection 24 generally arranged in another of the two end portions of the slot 15. As can be seen when comparing FIGS. 1-3, as the axial distance between the walls 13 and 22 changes, a depth of penetration will change correspondingly. Because the wall 13 serves as the maximum travel position of the lancet (this maximum being defined by contact between the lancet body and the inner surface of the wall 13) and the wall 22 is in contact with the skin, when the space between these walls 13 and 22 increases, the needle will penetrate less than when the space between these walls 13 and 22 increases. The maximum change is depth of penetration is thus determined by the maximum travel of the projection 24 in the slot 15. More specifically, because it is the cap 20 that rotates relative to the member 10 (when the cap assembly 100 is installed) during depth adjustment, the maximum change is depth of penetration adjustment is determined by the maximum travel of the slot 15 in relation to the projection 24, i.e., between the positions shown in FIGS. 2 and 3.

FIG. 4 shows the cap assembly 100 installed, e.g., removably installed, on a body or housing 210 of a lancet device 200. As is the case with typical lancet devices, the lancet device 200 can include a movable lancet holding member 220 which has a lancet receiving end receiving therein a lancet L. The lancet L has a body B and a needle N and can be of any known type, but preferably is a lancet having a synthetic resin body B and a needle that is at least one of about 3.5 mm long and greater than 3.5 mm long.

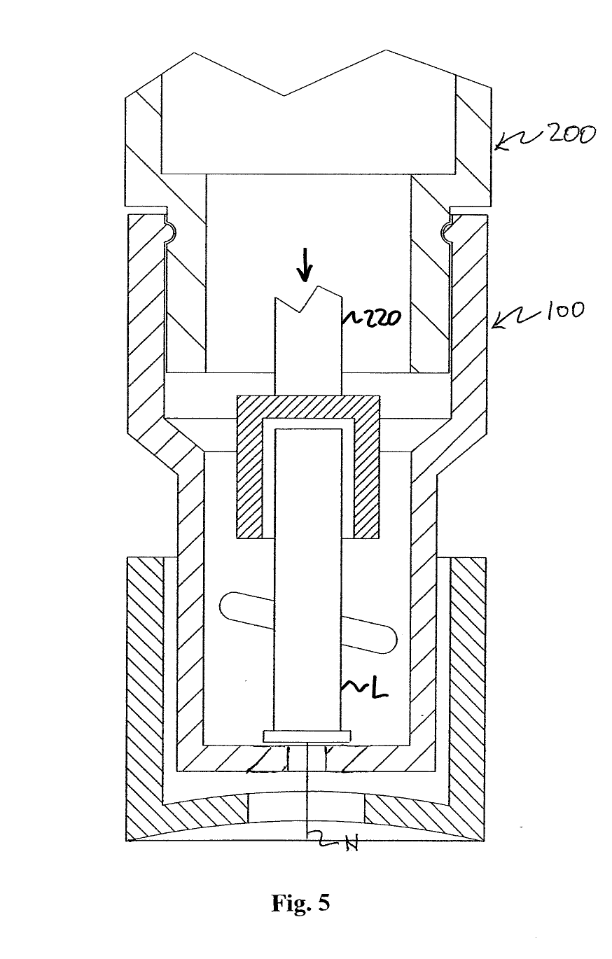

As can be seen in FIG. 5, the lancet L can be moved to the puncturing position by moving the holding member 220. This can occur when the user cocks and triggers the lancet device in a manner similar to that described in U.S. Pat. No. 4,976,724 to NIETO et al., the disclosure of which is hereby expressly incorporated by reference in its entirety.

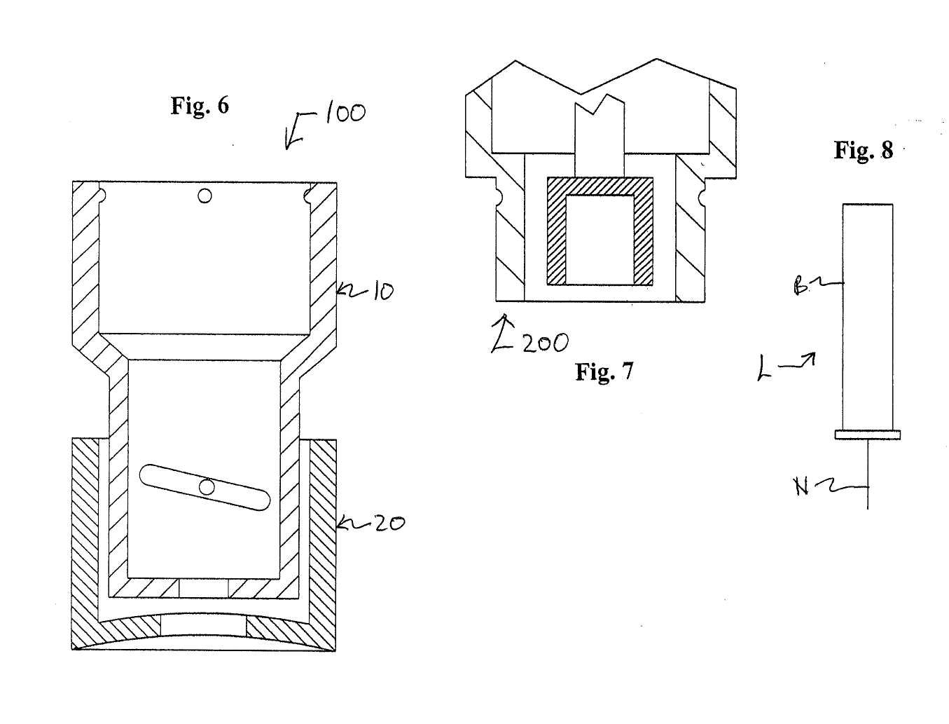

As can be seen in FIGS. 6-8, the cap assembly 100 can be removed from the lancet device 200, and after doing so, a user can remove the lancet L. After removal, a new lancet L can be installed on the holding member of the lancet device 200 and following by re-installation of the cap assembly 100.

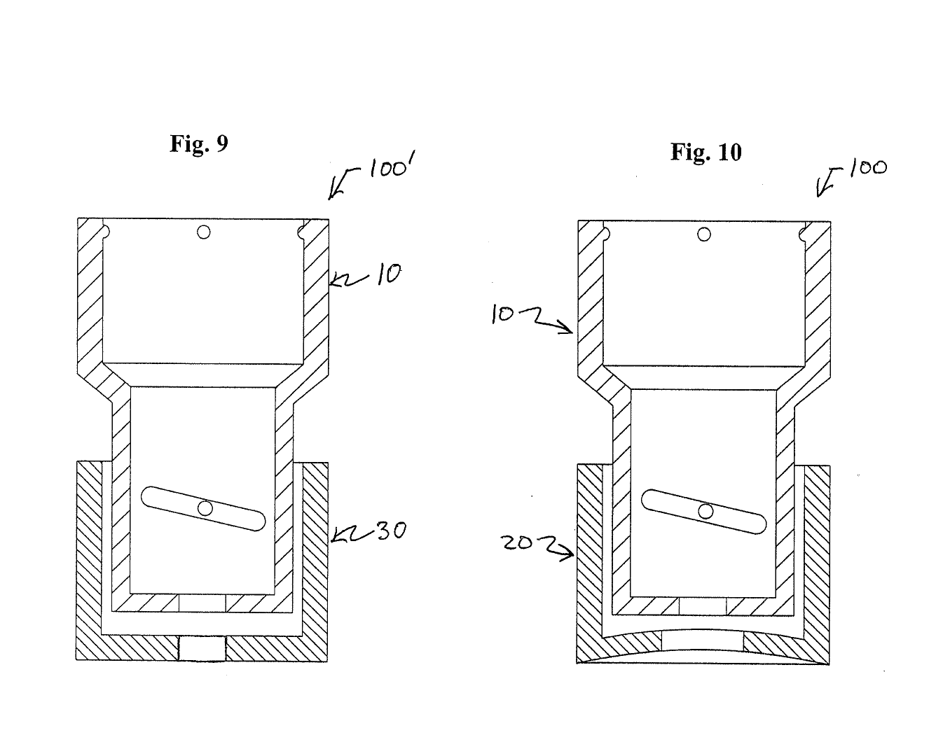

With reference to FIGS. 9 and 10, there is shown another embodiment of the invention. In this embodiment, one can utilize one of two or more removable cap assemblies as shown in FIGS. 9 and 10. The cap assembly 100′ of FIG. 9 can be used on a lancet device when the user wants to puncture skin using a non-AST cap. However, the cap assembly 100 of FIG. 10 (and also FIGS. 1-8) can be used on the lancet device when the user wants to puncture skin using an AST cap. Both cap assemblies 100 and 100′ utilize depth of penetration adjustment—the difference being that the alternative cap assembly 100′ has a generally planar skin contacting surface and/or can be of the standard or non-AST type and/or is designed for puncturing skin on, e.g., a finger tip.

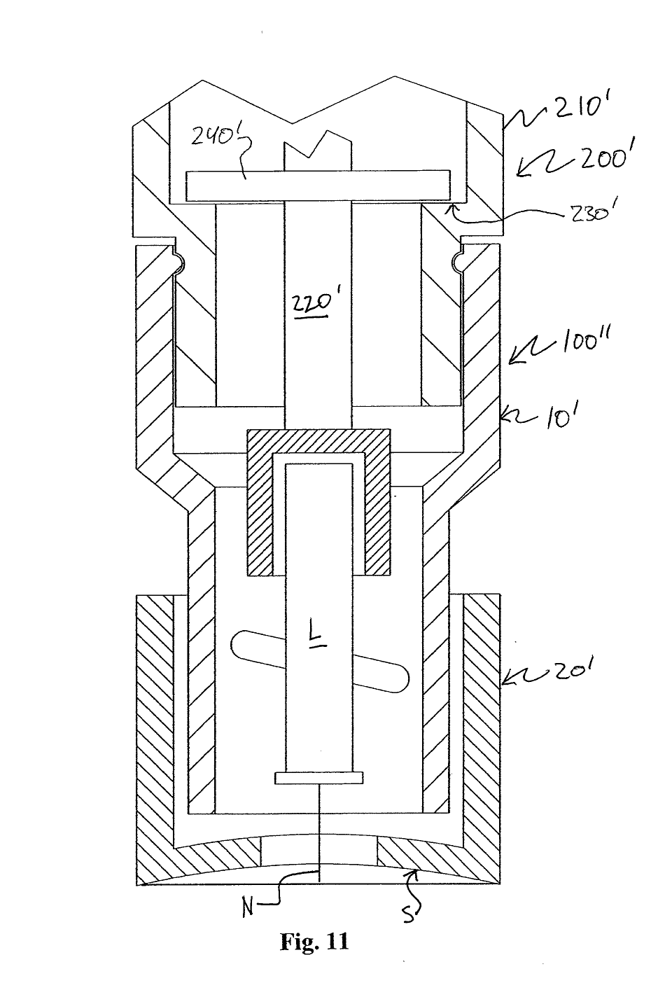

FIG. 11 shows another embodiment of a cap assembly 100″ installed, e.g., removably installed, on a body or housing 210′ of a lancet device 200′. In this embodiment, the lancet device 200′ includes a movable lancet holding member 220′ which has a lancet receiving end receiving therein a lancet L, and also a stop shoulder or flange 240′ which is sized and configured to contact a stop shoulder 230′ of the body 210′. Contact between the surfaces of the stop shoulders 240′ and 230′ limits depth of penetration movement of the holding member 220′ when it is caused to move by a spring (not shown). However, depth of penetration, i.e., the amount the needle N of the lancet L penetrates skin, is determined by the relative position of the skin contacting surface S in relation to the surface 230′. To adjust the penetration depth, the user rotates the cap 20′ in opposite directions (while the member 10′ remains non-rotatably connected to the body 210′) with rotation in one direction increasing the distance between the skin contacting surface S and the surface 230′ and rotation in another direction decreasing the distance between the skin contacting surface S and the surface 230′. As was the case with the previous embodiment, the lancet L has a body B and a needle N and can be of any known type, but preferably is a lancet having a synthetic resin body B and a needle that is at least one of about 3.5 mm long and greater than 3.5 mm long.

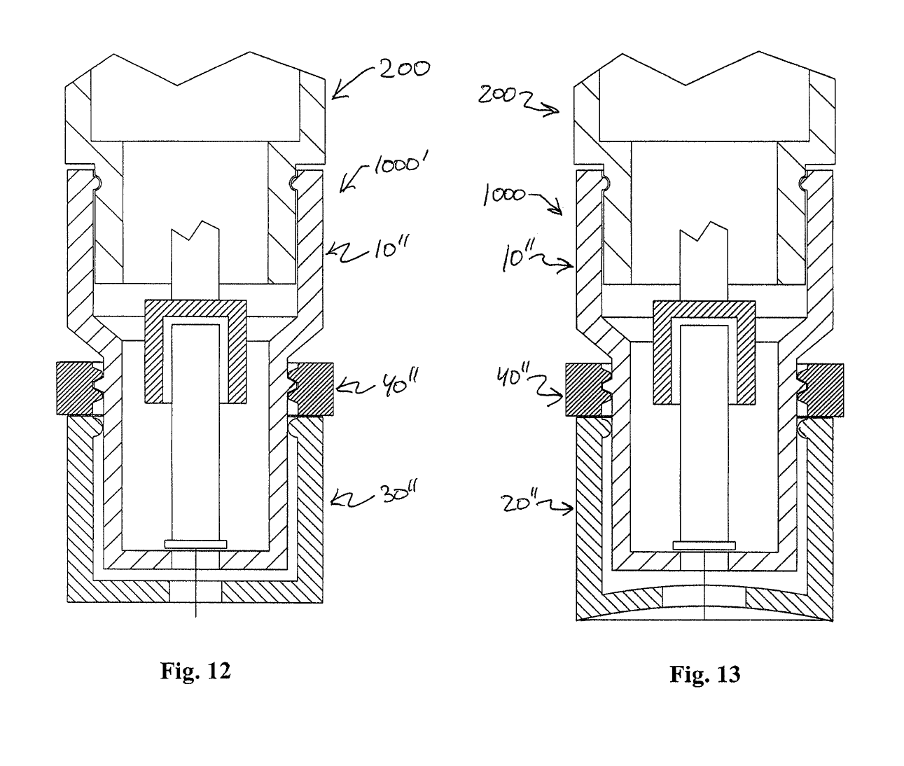

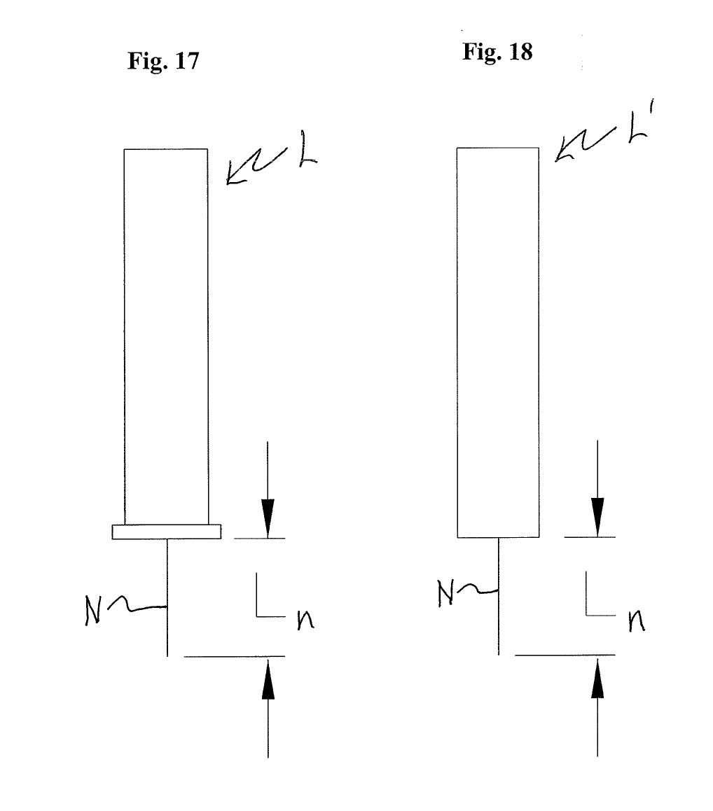

With reference to FIGS. 12-16, there is shown another embodiment of the invention. In this embodiment, one can utilize one of two or more removable cap assemblies as shown in FIGS. 12 and 13 on the lancet device 200 similar to that shown in FIG. 4. The cap assembly 1000′ of FIG. 12 can be used on a lancet device 200 when the user wants to puncture skin using a non-AST cap 30″. However, the cap assembly 1000 of FIG. 13 can be used on the lancet device 200 when the user wants to puncture skin using an AST cap 20″. Both cap assemblies 1000 and 1000′ utilize depth of penetration adjustment—the difference being that the alternative cap assembly 1000′ has a generally planar skin contacting surface and/or can be of the standard or non-AST type and/or is designed for puncturing skin on, e.g., a finger tip. Moreover, the cap assemblies 1000 and 1000′ have features in common such as the member 10″ and the depth adjuster ring 40″. Indeed, these parts can remain connected to the lancet device 200 while the user changes the non-AST cap 30″ for the AST cap 20″ and vice versa. In embodiments, the caps 30″ and 20″ are configured so that, when the ring 40″ is in a particular depth of penetration setting, replacing the non-AST cap 30″ with the AST cap 20″ results in the same puncturing depth, i.e., the indented surface of the AST cap 20″ is taken into account. In other embodiments, the caps 30″ and 20″ are configured so that, when the ring 40″ is in a particular depth of penetration setting, replacing the non-AST cap 30″ with the AST cap 20″ results in different puncturing depths, i.e., the indented surface of the AST cap 20″ is not taken into account and the puncture is deeper with the AST cap 20″, though not necessarily more painful because the AST cap 20″ is typically used on skin having a lower density of nerve endings.

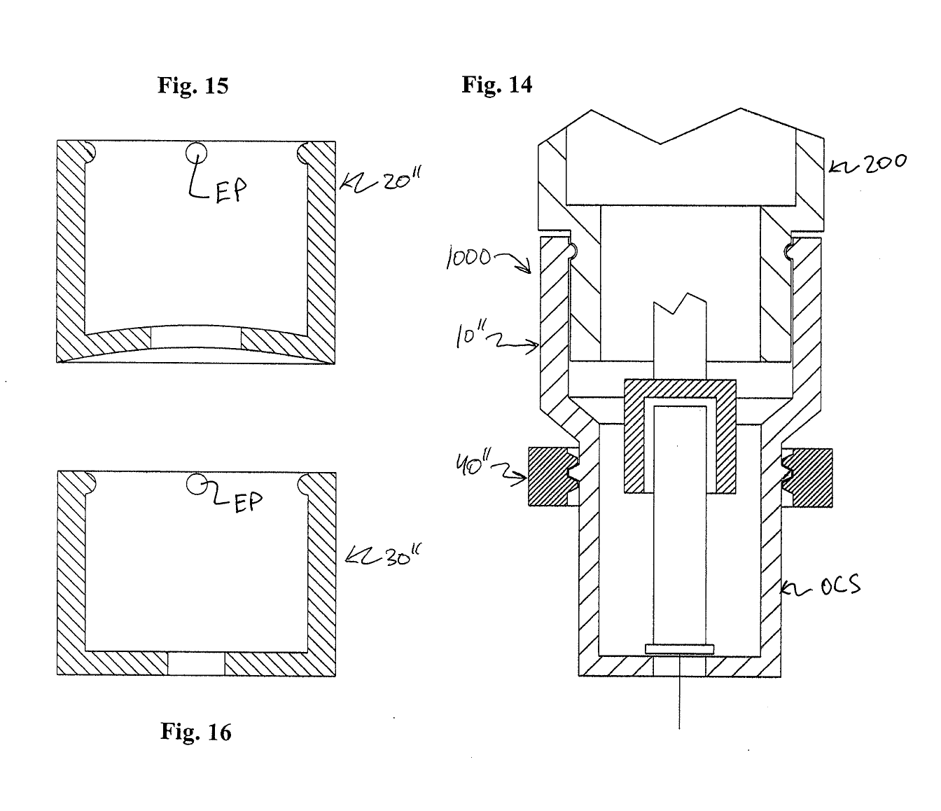

With reference to FIGS. 14-16, it can be seen how the parts 10″ and 40″ can remain connected to the lancet device 200 while the user can install and change the non-AST cap 30″ for the AST cap 20″ and vice versa. In embodiments, the caps 30″ and 20″ have the same distal end areas with internal engaging projections EP that frictionally engage with an outer cylindrical surface OCS of the member 10″. This engagement allows the caps 30″ and 20″ to remain connected to the member 10″ until the user slides them off. The depth adjustment results from the threaded connection between the ring 40″ and the member 10″ and specifically between an internal thread of the ring 40″ engaging with an external thread of the member 10″. An advantage of this embodiment relates to the fact that one can replace the caps 20″ and 30″ without removing the main components of the front cap assembly 1000.

FIGS. 17 and 18 show different configurations of lancets L and L′ which can be utilized with the cap assemblies and lancet devices of the invention and shows how the length Lₙ of the lancet needle N is measured. In the example of FIG. 17, the length Lₙ of the lancet needle N is measured from a stop shoulder surface, i.e., the surface which contacts a stop surface of the cap assembly as shown in FIG. 5, of the lancet body. In the example of FIG. 18, the length Lₙ of the lancet needle N is measured from an end surface of the lancet body. The lancet L′ of FIG. 18 is more properly usable in embodiments (such as FIG. 11) which do not rely on contact between the lancet and a surface of the lancet device of cap assembly.

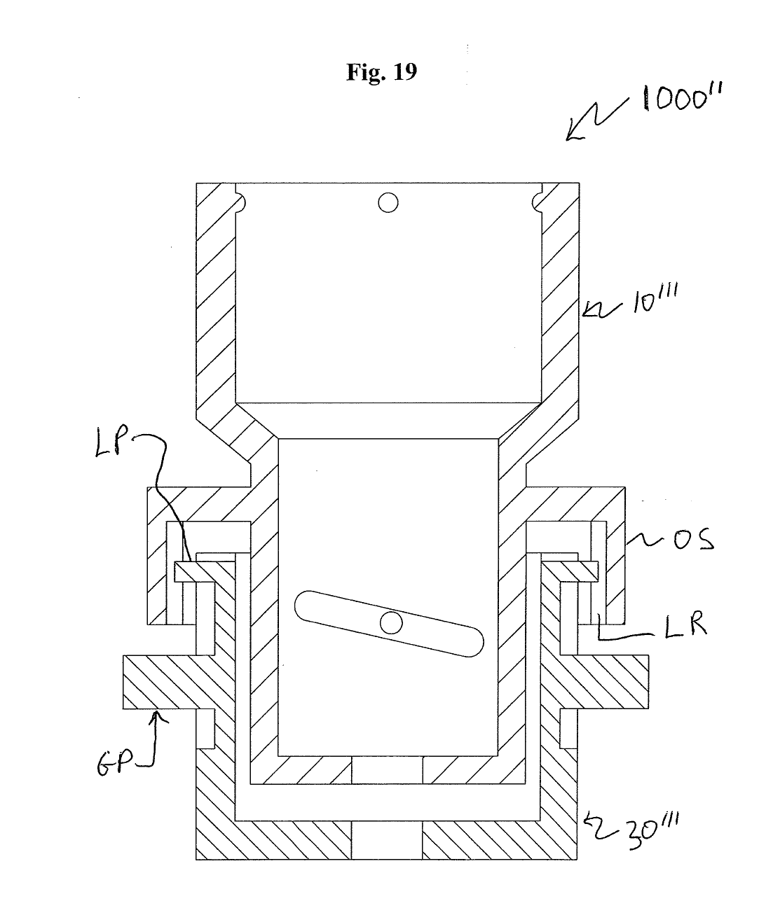

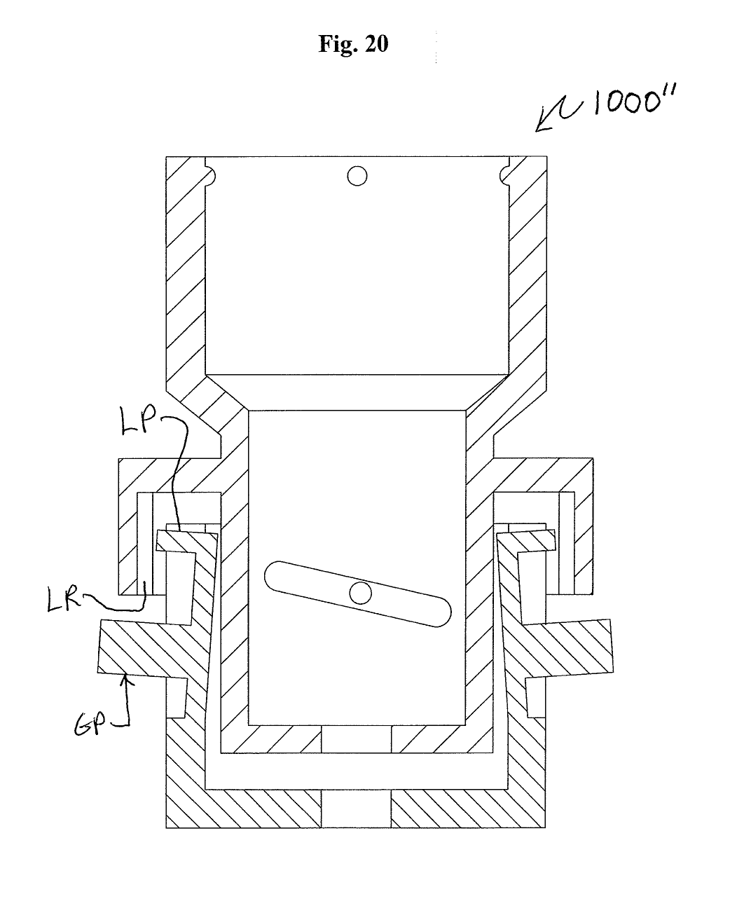

With reference to FIGS. 19-22, there is shown another embodiment of the invention. In this embodiment, a removable cap assembly 1000″ shown in FIG. 19 can be installed on a lancet device similar to that shown in FIG. 4. The cap assembly 1000″ can be used when the user wants to puncture skin using a non-AST cap 30′″. The cap assembly 1000″ utilizes depth of penetration adjustment, but also with locking positions and has a generally planar skin contacting surface. Moreover, the cap assembly 1000″ is an assembled unit utilizing the member 10′″ and the cap 30″. Depth adjustment results from the locking interaction between locking recesses LR of an outer cylindrical flange OS and one or more releasable locking projections LP arranged on one or more deflectable portions having a gripping projection GP. In embodiments, the cap 30′″ is configured so that, when in a particular depth of penetration setting, the non-AST cap 30′″ remains locked in this position. To change the depth of penetration setting, the user pushes in on the gripping projections GP as shown in FIG. 20 which causes the locking projections LP to come out of locking engagement with locking recesses LR. The user can then rotate the cap 30′″ to a different depth of penetration position.

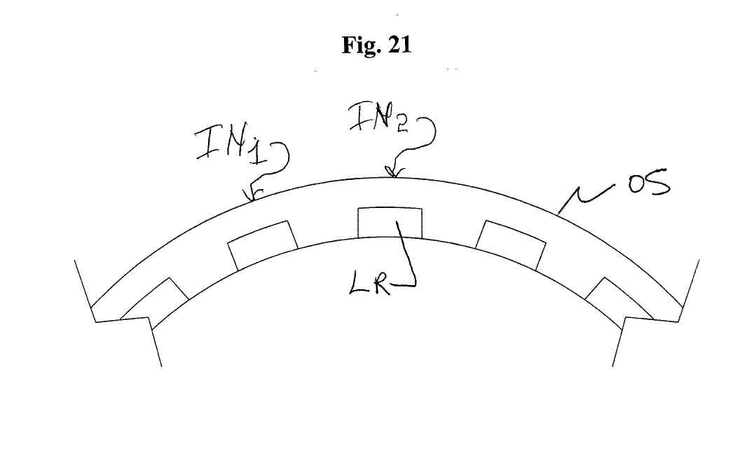

As is apparent from FIG. 21, a locking recess LR can be arranged in each rotation position for which there is an indicator or indicia. Thus, for example, when one of the locking projections LP engages with one locking recess LR corresponding to indicia IN₁, the needle N will penetration a different amount than when the same locking projection LP engages with a different locking recess LR corresponding to indicia IN₂. Also apparent from FIG. 21, the locking recesses LR are axial grooves formed in the inner circumferential surface of the outer surface portion OS while the indicia IN₁ and IN₂ are arranged on an outer circumferential surface of the outer surface portion OS.

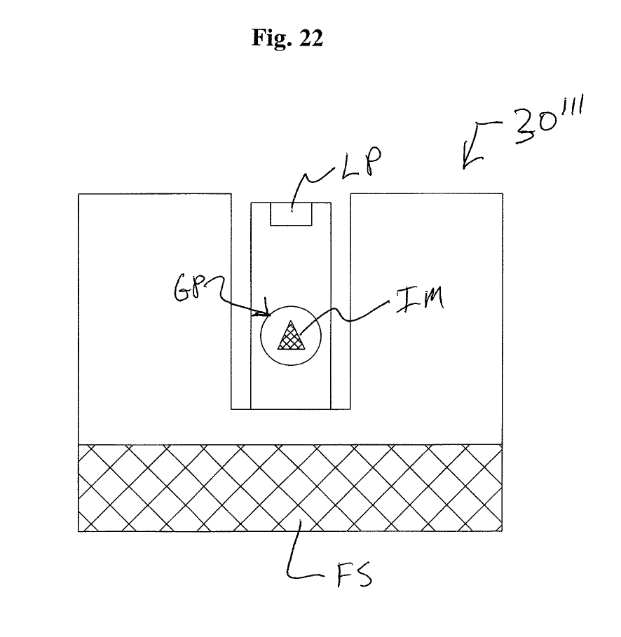

As is apparent from FIG. 22, the cap 30′″ can utilize two oppositely arranged locking projections LP and gripping projections GP arranged on a deflectable portion of the wall of the cap 30′″. Each gripping projection GP can also include an indicator or indicia IM (e.g., a triangle or arrowhead) which, depending on the rotational position of the cap 30′″, will be located adjacent a respective indicia IN₁ or IN₂, etc., thereby indicating which axial slot or locking recess LR is being engaged with one of the locking projections LP.

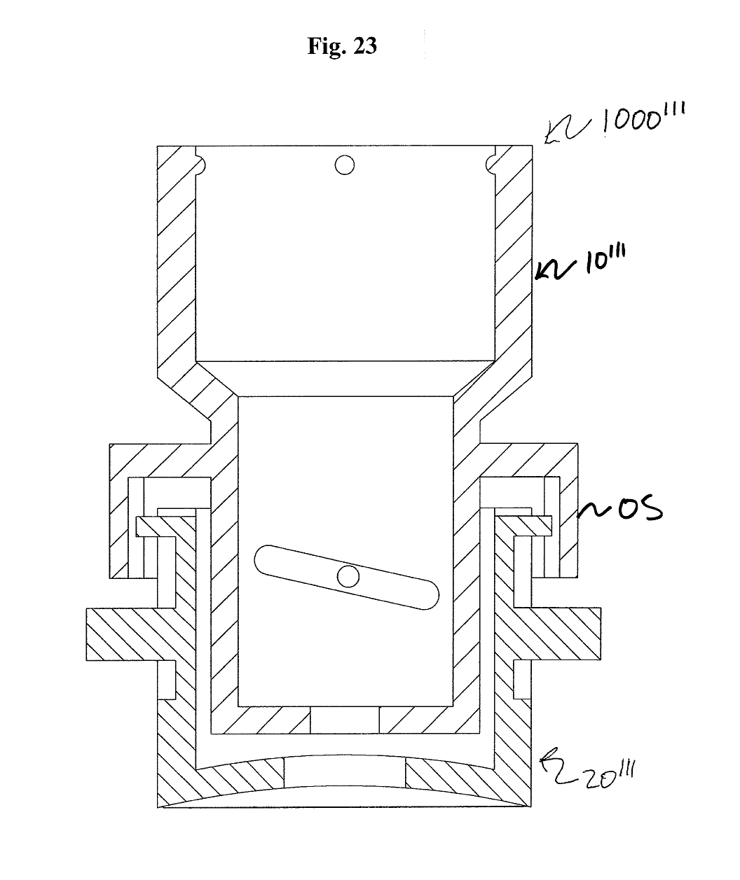

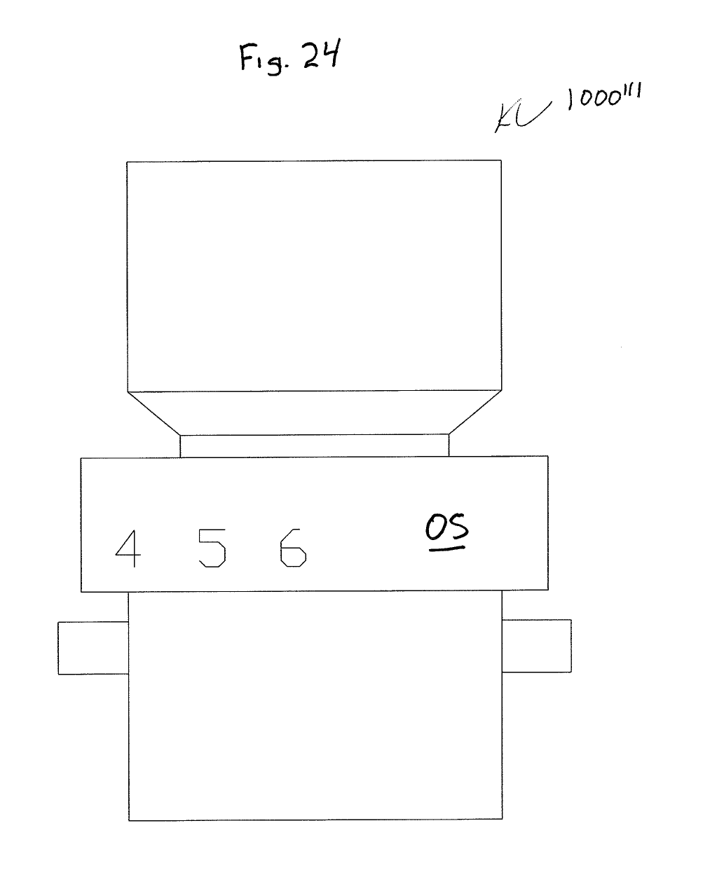

With reference to FIGS. 23 and 24, there is shown another embodiment of the invention. This cap assembly 1000′″ is similar to that of FIGS. 19-22 except that the removable non-AST cap assembly 30′″ is replaced with an AST cap 20′″. As such, the cap assembly 1000′″ utilizes depth of penetration adjustment, but also with locking positions and has a generally inwardly curved skin contacting surface. Moreover, the cap assembly 1000′″ is an assembled unit utilizing the member 10′″ and the cap 20′″. Depth adjustment results from the locking interaction between locking recesses of an outer cylindrical flange OS and one or more releasable locking projections arranged on one or more deflectable portions having a gripping projection. In embodiments, the cap 20′″ is configured so that, when in a particular depth of penetration setting, the AST cap 20′″ remains locked in this position. To change the depth of penetration setting, the user pushes in on the gripping projections (similar to that shown in FIG. 20) which causes the locking projections to come out of locking engagement with locking recesses. The user can then rotate the cap 20′″ to a different depth of penetration position and allow the locking recesses to automatically engage with other locking recesses. FIG. 24 shows one example of indicia that can used on the outer cylindrical flange OS.

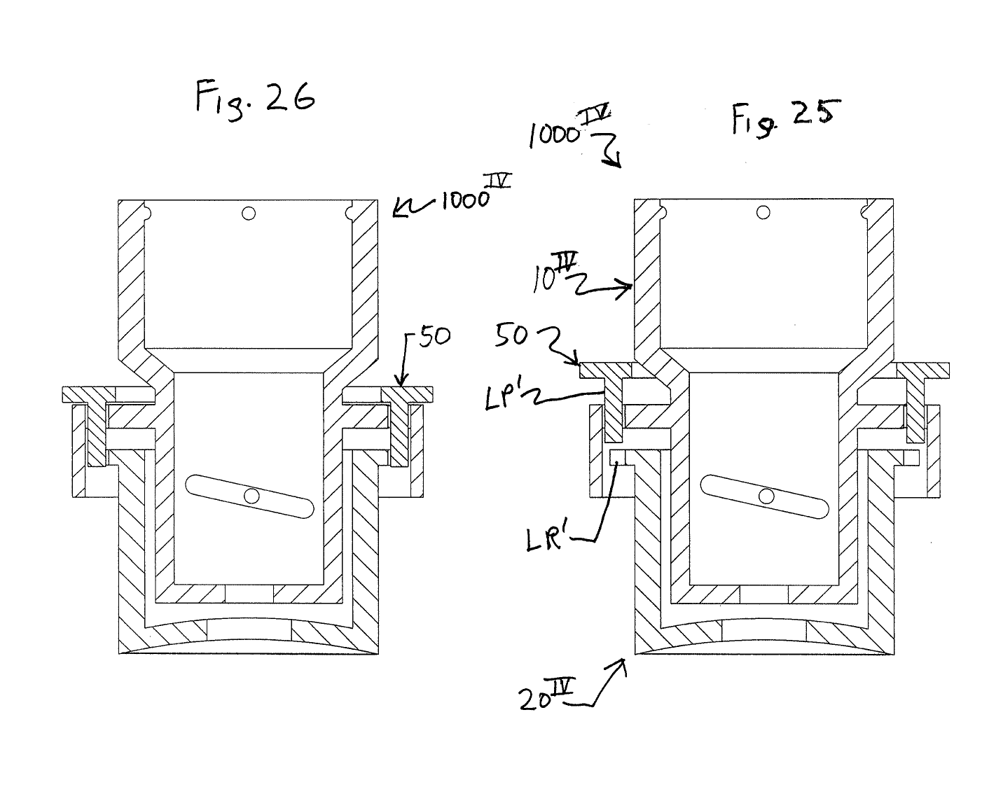

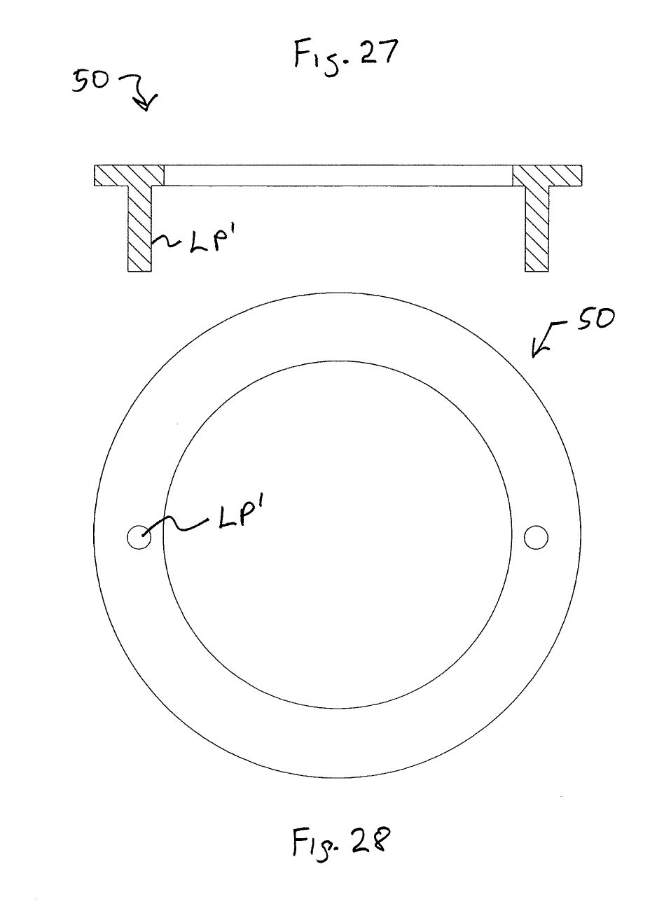

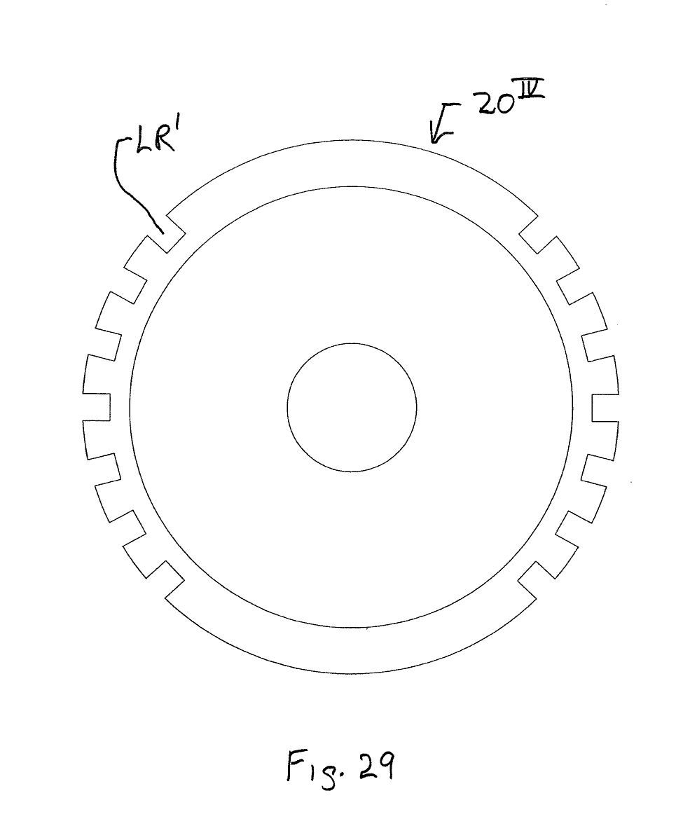

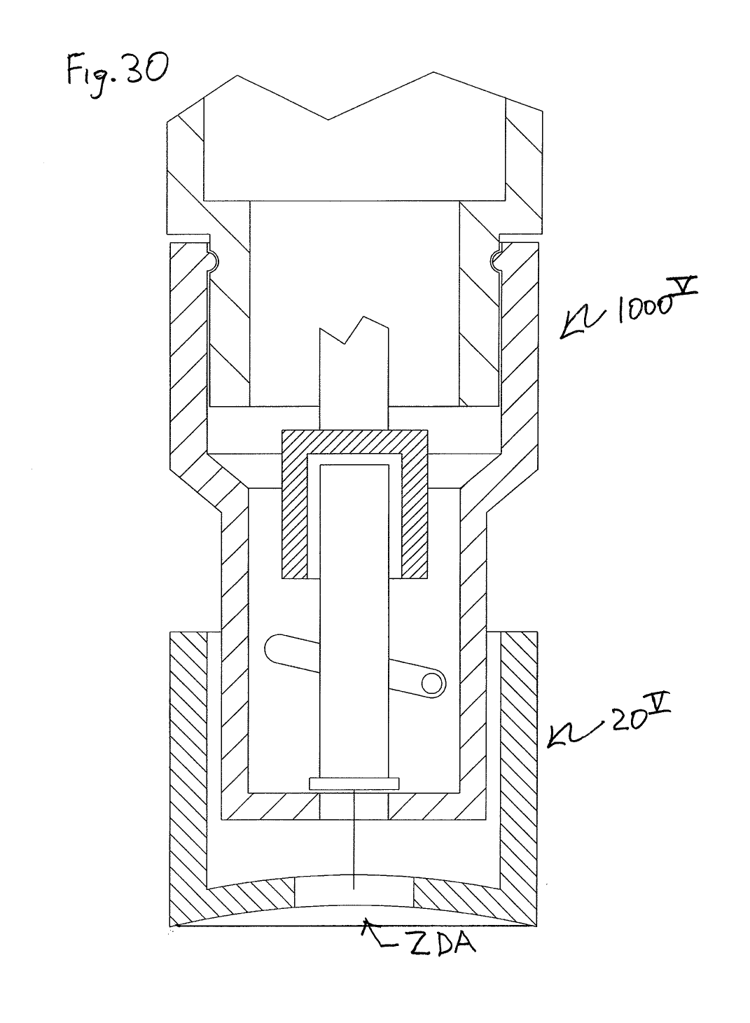

With reference to FIGS. 25-29, there is shown another embodiment of the invention. In this embodiment, a removable cap assembly 1000^{IV} shown in FIGS. 25 and 26 can be installed on a lancet device similar to that shown in FIG. 4. The cap assembly 1000^{IV} can be used when the user wants to puncture skin using an AST cap 20^{IV}. The cap assembly 1000^{IV} utilizes depth of penetration adjustment, and also utilizes locking positions and has a generally inwardly curved skin contacting surface. Moreover, the cap assembly 1000^{IV} is an assembled unit utilizing the member 10^{IV}, a locking member 50 and the cap 20^{IV}. Depth adjustment occurs when a user rotates the cap 20^{IV} relative to the member 10^{IV} or vice versa. This rotation movement in opposite directions is regulated by engagement between one or more inwardly projecting circumferential projections and an elongated and angled slot in a manner similar to previous embodiments. Once in a desired depth of adjustment position, the user can activate the locking system to maintain this position. The locking system results from the locking interaction between locking recesses LR′ of an outer flange arranged on the cap 20^{IV} and one or more releasable locking projections LP′ arranged on an axially movable locking member 50. In embodiments, the cap 20^{IV} is configured so that, when in a particular depth of penetration setting and when the member 50 is moved to the locked position shown in FIG. 26 from the unlocked position shown in FIG. 25, the AST cap 20^{IV} remains locked in this position. To change the depth of penetration setting, the user moves the locking member 50 back from the position shown in FIG. 26 to the position shown in FIG. 25 which causes the one or more locking projections LP′ to come out of locking engagement with the one or more locking recesses LR′. The user can then rotate the cap 20^{IV} to a different depth of penetration position relative to the member 10^{IV} or vice versa. To then lock the depth of penetration setting, the user moves the locking member 50 from the position shown in FIG. 25 forward and/or to the position shown in FIG. 26 which causes the one or more locking projections LP′ to reach a locking engagement with the one or more locking recesses LR′. Although not shown, a shoulder or flange can be arranged on the member 10^{IV} to axially retain the member 50 from a side opposite the side having the locking projections LP′. A spring or compressible material or compressible O-ring (not shown) can also be utilized to bias toward, or retain the member 50 in, the unlocked position shown in FIG. 25, or, more preferably, in the locked position shown in FIG. 26. In the latter case, the spring or compressible material or ring would be arranged between a flange or shoulder (not shown) of the member 10^{IV} and the side of the member 50 opposite the locking projections LP′.

With reference to FIG. 30, there is shown another embodiment of the invention. In this embodiment, a removable cap assembly 1000^{V} similar to that of FIG. 10 utilizes an AST cap 20^{V} that can be rotated to a depth adjustment position which ensures that no puncturing of the skin can occur. This setting can be considered a “zero” setting and functions as a safety system. As is notable in FIG. 30, in this setting, the puncturing end of the needle of the lancet cannot extend out past the skin contacting surface of the cap 20^{V} and, in fact, never reaches to the surface when moved to a puncturing position and/or a maximum extended position. This setting can thus be designated a zero depth adjustment ZDA setting. A mechanism (not shown) can be used to releasably retain the cap 20^{V} in this position as well as in all of the depth of penetration puncturing positions. This depth setting retaining arrangement can preferably be utilized in all other embodiments disclosed herein. Such retaining mechanisms are well known and are therefore not more fully described herein. Reference, however, is made to U.S. Pat. No. 5,613,978 to HARDING and to U.S. Pat. No. 6,558,402 to CHELAK et al. for teaching non-limiting examples of such mechanisms, the disclosure of each of these documents is herein expressly incorporated by reference in their entireties. With the embodiment of FIG. 30, even if the device is accidentally triggered, it cannot puncture the user's skin while in the safety position.

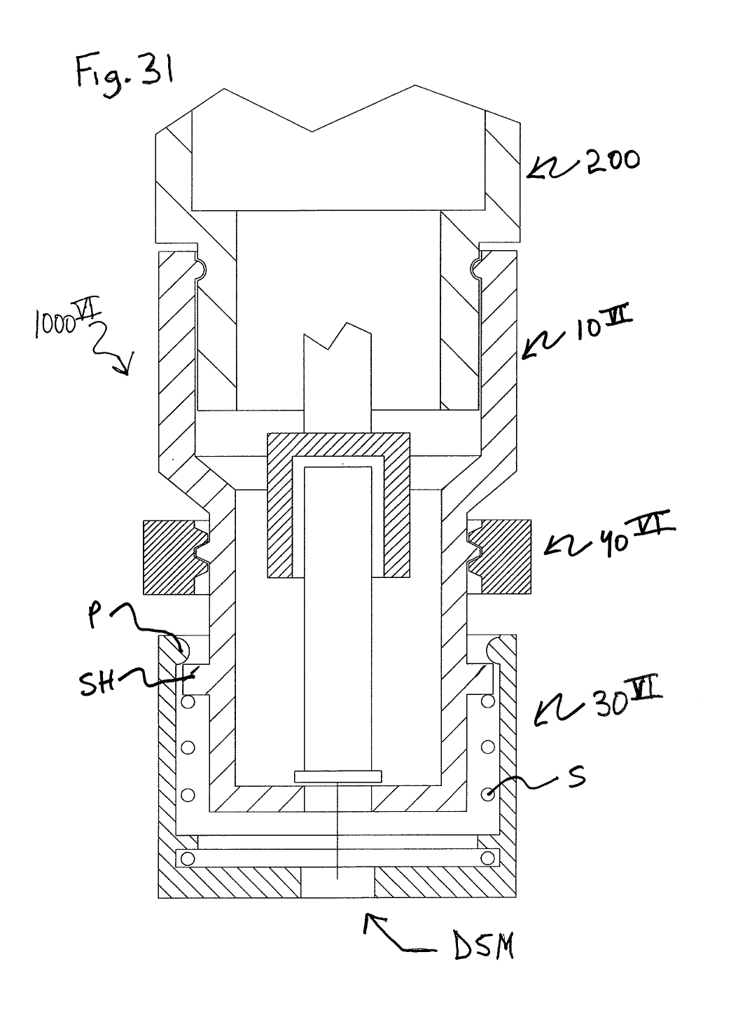

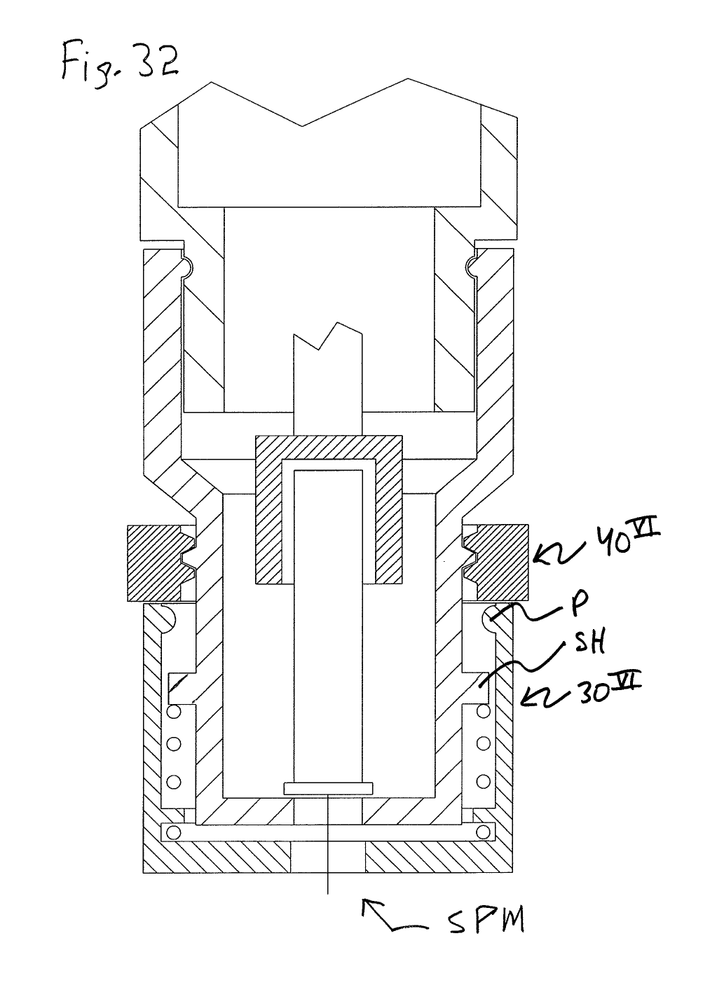

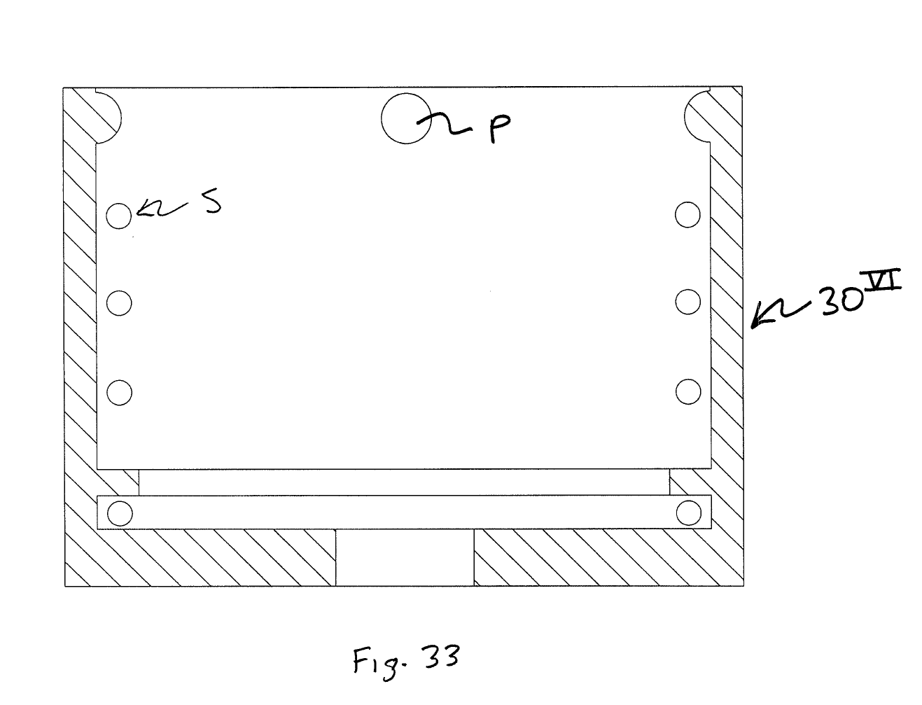

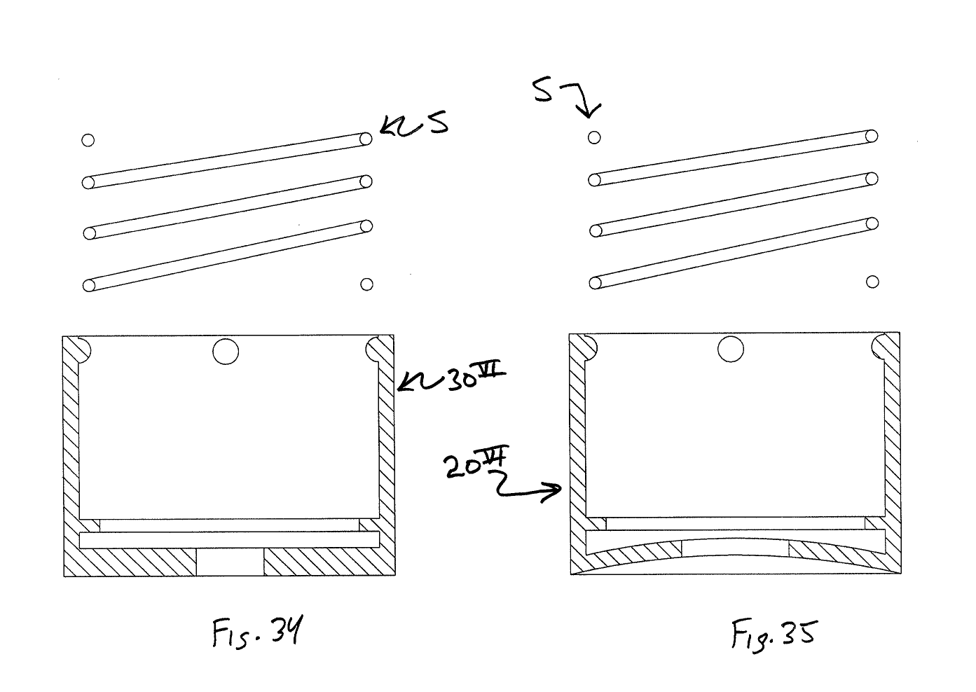

With reference to FIGS. 31-35, there is shown still another embodiment of the invention. In this embodiment, one can also utilize one of two or more removable cap assemblies with one of the cap assemblies being shown in FIGS. 31 and 32 arranged on the lancet device 200 similar to that shown in FIG. 4. The cap assembly 1000^{VI} of FIGS. 31 and 32 can be used on a lancet device 200 when the user wants to puncture skin using a non-AST cap 30^{VI}. However, a cap assembly similar to that shown in FIGS. 31 and 32, but utilizing the AST cap 20^{VI} shown in FIG. 35, can be used on the lancet device 200 when the user wants to puncture skin using an AST cap 20^{VI}. Each cap assembly utilizes depth of penetration adjustment—the difference being that the alternative cap assembly 1000^{VI} has a generally planar skin contacting surface and/or can be of the standard or non-AST type and/or is designed for puncturing skin on, e.g., a finger tip. Moreover, if the cap 30^{VI} is made removable and replaceable with the cap 20^{VI}, the cap assemblies have common features such as the member 10^{VI} and the depth adjuster ring 40^{VI}. Indeed, in this modification, these parts can remain connected to the lancet device 200 while the user changes the non-AST cap 30^{VI} for the AST cap 20^{IV} and vice versa. In embodiments, however, the cap 30^{VI} is not removable from the member 10 and is instead biased toward the position shown in FIG. 31 via a spring S. The spring S functions as a safety mechanism such that if the device 200 is mistakenly triggered, the needle of the lancet will not extend out past the surface DSM. However, when the cap 30^{IV} is pressed against a skin surface by an amount sufficient to axially compress the spring S and so that it abuts or contacts the ring 40^{IV}, triggering of the device will result in a skin puncture of a depth determined by the adjusted or adjustment position of the ring 40^{VI}. An advantage of this embodiment is that adjustment can occur while the cap 30^{VI} is in the position shown in FIG. 31. To ensure that the cap 30^{VI} remains coupled to the member 10^{VI}, the cap 30^{VI} utilizes one or more projections P which contact a circumferential shoulder SH of the member 10^{VI}. In an embodiment similar to that of FIG. 31, but utilizing the cap 20^{VI} instead of the cap 30^{VI}, it would function in a similar manner.

According to non-limiting embodiments of the invention, there is provided a lancet device comprising a cap assembly (100) comprising a member (10) capable of being removably mounted to a body of the lancet device and an alternate site testing (AST) cap (20) mounted to the member (10). A lancet (L) is utilized having a needle (N) that is at least 3.5 mm long.

According to non-limiting embodiments of the invention, there is provided a lancet device comprising a cap assembly (100) one of comprising an alternate site testing (AST) cap (20) and a non-AST cap (30) each mountable to a member (10) and two separate caps (20, 30) each being installable on the lancet device wherein one of the caps is an AST cap (20) mounted to a member (10) and another of the caps is a non-AST cap (30) mounted to a member (10). The member (10) is capable of being removably mounted to a body (200) of the lancet device.

According to non-limiting embodiments of the invention, there is provided a lancet device comprising a cap assembly (100) comprising a member (10) capable of being removably mounted to a body (200) of the lancet device and an alternate site testing (AST) cap (20) capable of being mounted to the member (10). A non-AST cap (30) is utilized that is capable of being mounted to the member (10).

In embodiments, the invention provides for a cap assembly for a lancet device, wherein the cap assembly (100) comprises a member (10) capable of being removably mounted to a body (200) of the lancet device, an alternate site testing (AST) cap (20) capable of being mounted to the member (10), and a non-AST cap (30) capable of being mounted to the member (10).

In embodiments, the invention provides for a cap assembly for a lancet device comprising at least one of a member (10′″) capable of being removably mounted to a body of the lancet device and a cap (20′″/30′″) mounted to the member (10′″) and moveble between at least two locked positions and at least one cap (20′″/30′″) having at least two lockable depth of penetration adjustment positions. As used herein, the terms “lockable”, “locked”, “lock” or “locking” means at least one of the following in reference to a cap be in at least one depth of penetration position:

that a user cannot rotate the cap unless the user first unlocks a locking or retaining engagement so that he/she is then allows to rotate the cap; and/or

that the cap cannot rotate between different depth of penetration positions merely by rotating the cap such that some additional action, e.g., an unlocking action, must be taken other than merely rotating the cap; and/or

that even partial rotation of the cap is prevented until a locking mechanism is moved to an unlocking position; and/or

that even partial rotation of the cap is prevented when a locking mechanism is in a locked position and rotation cannot occur without injuring or destroying the locking mechanism or other element; and/or

that mere action or torque aiming to cause rotation of the cap is sufficient to unlock the cap or move it to another depth of penetration position; and/or

that a releasable locking mechanism is structured and arranged to prevent rotation of the cap until the locking mechanism is unlocked.

In embodiments, there is provided a cap assembly (1000′″) for a lancet device comprising at least one of a member (10^{IV}) capable of being removably mounted to a body of the lancet device and a cap (20^{IV}) mounted to the member (10^{IV}) and having at least the following modes of operation: an adjusting mode (FIG. 25) allowing a user to at least one of; adjust a depth of penetration position of a skin engaging surface of the cap, change an axial distance between a skin engaging surface of the cap, and move the cap between at least two depth of penetration positions; and a locking mode (FIG. 26) wherein the user is prevented from adjusting the depth of penetration until the user places the cap in the unlocked position.

In embodiments, there is provided a lancet device comprising a member (10^{IV}) capable of being mounted to the lancet device and a cap (20^{IV}) mounted to the member (10^{IV}) and the cap (20^{IV}) being movable between at least two depth of penetration positions when in an unlocked position (FIG. 25) and being prevented from moving between the at least two depth of penetration positions when in the locked position (FIG. 26).

In embodiments, there is provided a lancet device comprising at least two depth of penetration positions and is lockable in at least one of the at least two depth of penetration positions via a locking device.

In embodiments, there is provided a lancet device with depth of penetration adjustment and being lockable in at least one depth of penetration position via a locking device.

In embodiments, there is provided a lancet device with depth of penetration adjustment having at least one puncturing position and at least one non-puncturing position, e.g., a zero depth of adjustment (ZDA) position (see FIG. 30). In the non-puncturing position, the lancet needle moves to an extended position without puncturing a user's skin.

In embodiments, there is provided a lancet device comprising at least the following modes of operation: a mode (DSM) default setting mode wherein a skin engaging surface is automatically disposed in a position that prevents puncturing of a user's skin when the lancet device is triggered; and a mode (SPM) selective puncturing mode wherein the skin engaging surface is disposed in a position that allows puncturing of a user's skin when the lancet device is triggered.

In embodiments, there is provided a lancet device with depth of penetration adjustment comprising at least the following modes of operation: a mode DSM wherein a skin engaging surface is automatically disposed in a position that prevents puncturing of a user's skin when the lancet device is triggered; and a mode SPM wherein the skin engaging surface is disposed in a position that allows puncturing of a user's skin when the lancet device is triggered.

In embodiments, there is provided a lancet device comprising at least the following modes of operation: a mode DSM wherein the skin engaging surface is biased towards a position that prevents puncturing of a user's skin when the lancet device is triggered; and a mode SPM wherein the skin engaging surface is movable to a position that allows puncturing of a user's skin when the lancet device is triggered.

In embodiments, there is provided a lancet device comprising at least the following modes of operation: a default mode DSM wherein the skin engaging surface is in a position that prevents puncturing of a user's skin when the lancet device is triggered; and a puncturing mode SPM wherein the skin engaging surface is selectively movable to a position that allows puncturing of a user's skin when the lancet device is triggered.

In embodiments, there is provided a cap assembly for a lancet device comprising at least the following modes of operation: a default mode DSM wherein the skin engaging surface is in a position that prevents puncturing of a user's skin when the lancet device is triggered; and a puncturing mode SPM wherein the skin engaging surface is selectively movable to a position that allows puncturing of a user's skin when the lancet device is triggered.

In embodiments, the devices of anyone of the above-noted devices further comprise at least one of an alternate site testing (AST) cap, a non-AST cap, a depth of penetration adjustment system, and a locking system for locking the cap assembly in one of said modes of operation.

In embodiments, each lancet device embodiment disclosed in U.S. Pat. No. 5,464,418 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in U.S. Pat. No. 5,908,434 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in U.S. Pat. No. 6,022,366 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in U.S. Pat. No. 7,905,898 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in U.S. Pat. No. 7,621,931 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in US 2010/0274273 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in US 2008/0195132 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in US 2007/0083222 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.) shown and/or disclosed herein.

In embodiments, each lancet device embodiment disclosed in US 2006/0241668 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.,) shown and/or disclosed herein.

In embodiments, each lancet device embodiment disclosed in US 2006/0173478 to SCHRAGA, the entire disclosure of which is herein expressly incorporated by reference, is modified to utilize one or more of the cap assembly embodiments (100, 100′, 100″, 1000, 1000′, 1000″, 1000′″, 1000^{IV}, 1000^{V}, 1000^{VI} etc.,) shown and/or disclosed herein.

The devices described herein preferably utilize one or more features disclosed in prior art documents expressly incorporated by reference herein. This application and the documents expressly incorporated therein are hereby fully or entirely expressly incorporated by reference in the instant application. Furthermore, one or more of the various parts of the device can preferably be made as one-piece structures by e.g., injection molding, when doing so reduces costs of manufacture. Non-limiting materials for most of the parts include synthetic resins such as those approved for lancet devices, syringes, blood collection devices, or other medical devices. Furthermore, the invention also contemplates that any or all disclosed features of one embodiment may be used on other disclosed embodiments, to the extent such modifications function for their intended purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A lancet device comprising:
a cap assembly comprising:
a member capable of being removably mounted to a body of the lancet device; and
an alternate site testing (AST) cap mounted to the member; and
a lancet having a needle that is at least one of:
about 3.5 mm long; and
greater than 3.5 mm long.

2. The lancet device of claim 1, wherein the member comprises a stop surface adapted to be contacted by a front end of the lancet.

3. The lancet device of claim 1, wherein at least one of:
the AST cap is movable relative to the member between at least two axial positions and the at least two axial positions define different depth of penetrations of the needle;
the AST cap is movable relative to the member between plural axial positions; and
the AST cap is rotatable relative to the member between plural positions.

4. The lancet device of claim 1, wherein the lancet is removably mountable to a holding member of the lancet device.

5. The lancet device of claim 1, wherein the cap assembly utilizes depth of penetration adjustment.

6. The lancet device of claim 1, further comprising a second cap assembly adapted to be removably mounted to the body of the lancet device.

7. The lancet device of claim 6, wherein the second cap assembly utilizes depth of penetration adjustment.

8. The lancet device of claim 6, wherein the second cap assembly is a non-AST cap assembly.

9. The lancet device of claim 1, wherein the body of the lancet device utilizes depth of penetration adjustment.

10. The lancet device of claim 1, wherein the body comprises at least one of:
a stop surface for limiting forward travel of a lancet holding member movably arranged within the body; and
a stop surface for limiting forward travel of a lancet holding member movably arranged within the body.

11. The lancet device of claim 1, wherein the lancet device utilizes depth of penetration adjustment.

12. The lancet device of claim 11, wherein the depth of penetration adjustment is determined by a stop surface for limiting forward travel of a lancet holding member and a skin engaging surface of the AST cap.

13. The lancet device of claim 11, wherein the AST cap is adjustably movable between at least two depth of penetration positions, a first of the at least two depth of penetration positions being spaced from the stop surface by a lesser amount than a second of the at least two depth of penetration positions.

14. A lancet device comprising:
a cap assembly one of:
comprising an alternate site testing (AST) cap and a non-AST cap each mountable to a member; and
two separate caps each being installable on the lancet device wherein one of the caps is an AST cap mounted to a member and another of the caps is a non-AST cap mounted to a member,
wherein the member is capable of being removably mounted to a body of the lancet device.

15. The lancet device of claim 14, further comprising a lancet having a needle that is more than 3.3 mm long.

16. The lancet device of claim 14, wherein the member comprises a stop surface adapted to be contacted by a front end of the lancet.

17. The lancet device of claim 14, wherein at least one of:
the AST cap is movable relative to the member between at least two axial positions and the at least two axial positions define different depth of penetrations of the needle;
the AST cap is movable relative to the member between plural axial positions; and
the AST cap is rotatable relative to the member between plural positions.

18. The lancet device of claim 14, wherein the lancet is removably mountable to a holding member of the lancet device and the cap assembly utilizes depth of penetration adjustment.

19. The lancet device of claim 14, wherein the body of the lancet device utilizes depth of penetration adjustment.

20. A method of using the lancet device of claim 1, the method comprising:
mounting the member to a body of the lancet device;
placing the alternate site testing (AST) cap against a skin surface; and
forming a puncture in the skin surface with the needle.

21. A method of making the lancet device of claim 1, the method comprising:
mounting the member to a body of the lancet device; and
mounting the alternate site testing (AST) cap onto the member.

22. A cap assembly for a lancet device, the cap assembly comprising:
a member capable of being removably mounted to a body of the lancet device;
an alternate site testing (AST) cap capable of being mounted to the member; and
a non-AST cap capable of being mounted to the member.

23. The cap assembly of claim 22, further comprising a lancet having a needle that is more than 3.3 mm long.

24. A cap assembly for a lancet device comprising at least one of:
a member capable of being removably mounted to a body of the lancet device and a cap mounted to the member and moveble between at least two locked positions; and
at least one cap having at least two lockable depth of penetration adjustment positions.

25. A lancet device comprising at least one of:
a member capable of being removably mounted to the lancet device and a cap mounted to the member and moveble between at least two locked positions; and
a mechanism for locking a cap in at least two depth of penetration adjustment positions.

26. A cap assembly for a lancet device comprising at least one of:
a member capable of being removably mounted to a body of the lancet device and a cap mounted to the member and having at least the following modes of operation:
an adjusting mode allowing a user to at least one of:
adjust a depth of penetration position of a skin engaging surface of the cap;
change an axial distance between a skin engaging surface of the cap; and
move the cap between at least two depth of penetration positions; and
a locking mode wherein the user is prevented from adjusting the depth of penetration until the user places the cap in the unlocked position.

27. A lancet device comprising:
a member capable of being mounted to the lancet device and a cap mounted to the member; and
the cap being moveable between at least two depth of penetration positions when in an unlocked position and being prevented from moving between the at least two depth of penetration positions when in the locked position.

28. A lancet device with depth of penetration adjustment and being lockable in at least one depth of penetration position via a locking device.

29. A lancet device comprising at least the following modes of operation:
a mode wherein a skin engaging surface is automatically disposed in a position that prevents puncturing of a user's skin when the lancet device is triggered; and
a mode wherein the skin engaging surface is disposed in a position that allows puncturing of a user's skin when the lancet device is triggered.

30. A lancet device with depth of penetration adjustment comprising at least the following modes of operation:
a mode wherein a skin engaging surface is automatically disposed in a position that prevents puncturing of a user's skin when the lancet device is triggered; and
a mode wherein the skin engaging surface is disposed in a position that allows puncturing of a user's skin when the lancet device is triggered.

31. A lancet device comprising at least the following modes of operation:
a mode wherein the skin engaging surface is biased towards a position that prevents puncturing of a user's skin when the lancet device is triggered; and
a mode wherein the skin engaging surface is movable to a position that allows puncturing of a user's skin when the lancet device is triggered.

32. A lancet device comprising at least the following modes of operation:
a default mode wherein the skin engaging surface is in a position that prevents puncturing of a user's skin when the lancet device is triggered; and
a puncturing mode wherein the skin engaging surface is selectively movable to a position that allows puncturing of a user's skin when the lancet device is triggered.

33. A cap assembly for a lancet device comprising at least the following modes of operation:
a default mode wherein the skin engaging surface is in a position that prevents puncturing of a user's skin when the lancet device is triggered; and
a puncturing mode wherein the skin engaging surface is selectively movable to a position that allows puncturing of a user's skin when the lancet device is triggered.

34. The cap assembly of claim 33, further comprising at least one of:
an alternate site testing (AST) cap;
a non-AST cap;
a depth of penetration adjustment system; and
a locking system for locking the cap assembly in one of said modes of operation.
